(19) United States
(12) Patent Application Publication
(10) Pub. No.: US 2005/0210537 A1
(43) Pub. Date: Sep. 22, 2005

(54) Pluripotent mammalian cells

(76) Inventors: Tanja Dominko, Southbridge, MA (US); Raymond L. Page, Southbridge, MA (US); Alan Colman, Midlothian (GB); Todd Vaught, Christiansburg, VA (US); Vivienne Marshall, Christiansburg, VA (US)

Correspondence Address: KING & SPALDING LLP, 191 PEACHTREE STREET, N.E., 45TH FLOOR, ATLANTA, GA, 30303-1763, US

(21) Appl. No.: 11/131,703

(22) Filed: May 18, 2005

Related U.S. Application Data
(63) Continuation of application No. 09/881,204, filed on Jun. 15, 2001.
(60) Provisional application No. 60/211,593, filed on Jun. 15, 2000.

(51) Int. Cl. A01K 67/027; C12N 5/06; C12N 5/08

(52) U.S. Cl. 800/14; 800/15; 800/16; 800/17; 435/354; 435/364; 435/366

(57) ABSTRACT

The invention relates to a method of making pluripotent stem cells that does not involve the formation of early preimplantation embryos or fetal tissue. The method has general utility in the production of pluripotent stem cells from many mammalian species but has particular application in man where pluripotent stem cell production can be customized to particular human individual. The method involves the fusion of donor somatic or stem cells (or their karyoplasts) with cytoplasmic, membrane-delimited fragments of mammalian oocytes or zygotes. After the initial genomic reprogramming occurs, the cells can proliferate and thus multiply in vitro yielding a large number of autologous cells for cell therapy application. The result of this process is a cell population genomically identical to the somatic, differentiated cells derived from an individual patient. However, these cells are pluripotent in that upon application of specific growth factors, the cells are capable of differentiating into specific cell types as required by the sought clinical indication.

### CROSS-REFERENCE TO RELATED APPLICATIONS

The present application claims priority benefit to provisional U.S. Appl. No. 60/211,593, filed Jun. 15, 2000, which is herein incorporated by reference.

### BACKGROUND OF THE INVENTION

1. Field of the Invention

The present invention relates to the field of stem cells and pluripotent cells. Specifically, the invention relates to the production of pluripotent cells for transplantation and replacement of diseased or damaged tissue.

2. Related Art

The replacement of damaged organs and tissues is a major problem in health care. Most organs and tissues regenerate poorly in mammals and it is often not possible to repair damaged or diseased tissues with drugs. In a few cases, artificial materials, such as replacement joints or mechanical devices, such as renal dialysis machines, work well. Under other circumstances, organs or tissues from other individuals may be used. For instance, kidneys, hearts, and bone marrow, have been successfully transplanted. There are three major disadvantages to transplantation. One is the very limited supply of such organs and tissues, being largely dependent on post mortem donation from accident victims. Another is the high cost of treatment (for example, presently, it costs about $150,000 for a replacement heart). The third is the need to maintain recipients on immunosuppressive drugs to avoid rejection due to the genetic differences between donor and recipient. Though the supply problem could be solved by the use of organs obtained from non-human, species of a similar size and physiology (e.g. the pig), immuno-incompatibility still remains a major problem. Xenotransplantation also poses the danger of introducing new viruses which are pathogenic to humans and might emerge from long term association with an organ from a different species. For example, recent findings show that porcine endogenous retroviruses can infect human cells in vitro.

An alternative strategy is the use of “ready made” organs and tissues. Much recent interest has centered on stem cells to accomplish this (reviewed by Vogel, Science 283:1432-1434 (1999)). These cells display a unique capacity to self-renew, as well as to produce partially committed progenitor cells (reviewed by Fuchs and Segre, Cell 100:143-155 (2000); and by Weissman, Cell 100:157-168 (2000)). For example, mammalian bone marrow contains a range of hematopoietic (blood-forming) stem cells. This feature has been exploited clinically in bone marrow transplantation, by allowing these stem cells to repopulate once the diseased cells have been removed. With new in vitro culture techniques, there may be even more ways of manipulating these stem cells. For example, signaling molecules, such as interleukins, may be used to isolate certain hematopoietic stem cell populations which then might be induced to proliferate, providing enriched pools. Under appropriate culture conditions, these cells may mature into more restricted stem cell populations and differentiation factors applied to produce fully differentiated cells. In this way, factors such as erythropoietin and interleukins may be used to produce erythrocytes and granulocytes. When such populations of differentiated cells have been reproducibly generated they will be useful clinically for transplantation. Adult neural stem cells show particular promise in these applications because of their ability to proliferate in culture without loss of developmental potential. Such cells have been shown to restore neurological function in the mouse (Snyder et al., Adv. Neurol. 72:121-132 (1997) and rat (Zhang et al., Proc. Natl. Acad. Sci. USA 96:4089-4094 (1999)) central nervous systems.

Although they are not yet completely understood, the mechanisms by which stem cells are programmed to differentiate into different cell lineages may allow opportunities for manipulation. It has been observed that stem cells of one type may, in some instances, generate cells of a completely different lineage. Thus, neural stem cells can generate hematopoietic stem cells when transplanted into mice that have been irradiated to eliminate their own blood stem cells (Bjornson et al., Science 283:534-537 (1999)). Similarly, cells capable of generating functional astrocyte-like cells (Azizi et al., Proc. Natl. Acad. Sci. USA 95: 3908-3913 (1998); Kopen et al., Proc. Natl. Acad Sci. USA 96:10711-10716 (1999)) and muscle (Ferrari et al, Science 279:1528-1530 (1998)) have been reported in human bone marrow stromal cell preparations. From these observations, it appears that stem cells can be reprogrammed under the correct conditions.

The most versatile of the stem cells are mouse embryonic stem (ES) or embryonic germ (EG) cells. These cells are obtained from early mouse embryos or primordial germ cells, respectively. They proliferate well in culture and differentiate into adult cell types, (Evans et al., Nature 29:154-156 (1981); Matsui et al., Nature 353:750-751 (1991)) including the germ cells, when transplanted into host embryos. Transplanting the stem cells into a host embryo, thus propagates their genotype to succeeding generations. These cells, especially ES cells, have proved extremely valuable to basic and applied research. Gene manipulation techniques work particularly well with these cells and the addition of new, sometimes very large gene constructs, or the replacement and/or modification of endogenous genes can be affected with surprising ease (Bradley et al., Bio/Technology 10:534-539 (1992)). Most importantly, these modifications can be made without affecting the developmental potential of the cells so that new lines of transgenic mice can be made. The ability to perform subtle gene alterations or replacements would be extremely useful in livestock species and laboratory animals (in addition to mice).

Surprisingly, despite many attempts, cells with such properties have never been isolated from other, non-mouse mammalian species. Those “pluripotent” cells which have been described have never been shown to contribute successfully to the germ line (e.g. Notorianni et al., J. Reprod Fert. Suppl. 43:255-260 (1991); Saito, et al., Roux's Arch Dev. Biol. 201:134-141 (1992); Handyside, et al., Roux Arch Dev Biol 196:185-190 (1987); Cherny, et al., Theriogenology 41:175 (1994); Van Stekelenburg-Hamers et al., Mol. Reprod Dev. 40:444-454 (1995); Smith et al., WO 94/24274 (1994); Evans et al., WO 90/03432 (1990); Wheeler et al., WO 94/26889 (1994); Wheeler et al., WO 94/26884 (1994). Though there have been recent reports of human cells having several properties of ES cells (Thomson et al., Science 282:1145-1147 (1998); Reubinoff et al., Nature Biotechnology 18:399404 (2000)) and EG cells (Shamblott et al., Proc. Natl. Acad Sci. USA 95:13726-13731 (1998)), and of human ES cells being capable of forming neural cells in vitro (Reubinoff et al., supra), these cells must be obtained by killing early embryos, and so will always present ethical problems.

A major problem with the strategies discussed so far, including those that utilize human ES or EG cells, is that of immunological incompatibility. While this problem might be avoided by using donor tissue or stem cells from the same individual who is to receive the transplant, as in a skin transplant for burn patients, the amount of such tissue or stem cells is often very limited or impossible to obtain. An ideal solution would be to create the required stem cells from the somatic cells of the individual patient. Since these cells would be autologous, there would be no issues of rejection within that individual. Several routes for achieving this objective are described below. In general, the production of stem cells from an existing somatic cell will require reprogramming of a differentiated, adult cell.

The degree to which the differentiated, adult cell is reprogrammed must be considered. For example, adult stem cells have a more restricted developmental potential. They are multipotential, and thus are capable of being induced to differentiate along many, but not all, cell lineages characteristic of the adult animal. ES and EG cells are pluripotent and therefore capable of differentiating into many if not all the cell types characteristic of an adult (with the exception of trophectoderm tissue). Only the fertilized zygote, which can give rise directly to all cell types comprising the developing embryo and therefore the adult animal, is totipotent. To prepare adult, differentiated cells for use in transplantation to regenerate diseased tissues or organs, it is not necessary to produce a totipotent cell. Instead pluripotent cells have enough potential to be induced into any type of cell lineage needed.

The differentiated state in somatic cells is very stable due to dynamic interactions between components of the nucleus and those in the cytoplasm (Blau and Baltimore, J. Cell Biol. 112:781-783 (1991). This inhibits reprogramming of the nuclear genes. Reprogramming can be achieved, though, by exposing the nucleus to a new cytoplasm. Experimentally induced fusion of two different cell types has demonstrated nuclear reprogramming (for review, see Ringertz and Savage, Cell Hybrids, Academic Press (1976)). In these experiments, cells from different species are often used in order to provide suitable molecular markers.

After the fused cells, called heterokaryons, are formed, reprogramming of at least one nucleus usually occurs. This reflects the influence of trans-acting cytoplasmic factors from one of the original cells, causing the other nucleus to be reprogrammed. For example, fusion of an EG cell and a thymocyte caused several thymocyte specific genes to be down regulated, indicating a possible dominance over the nucleus of the differentiated thymocyte by the more pluripotent cytoplasm of the EG cell (Tada et al., EMBO J. 16:6510-6520 (1997). Unfortunately, heterokaryons are not an option for producing stem cells for transplantation because they do not divide, and therefore cannot be propagated into a sufficient number of cells. Instead, heterokaryons have a variable and often reduced number of chromosomes from each donor, which mingle within the same nuclear membrane.

As an alternative to fusing complete cells, success has been reported in reconstructing cells after the fusion of cytoplasts and karyoplasts, each prepared from cultured, differentiated cells (Hightower et al., Proc Natl Acad Sci 80:5310-5314 (1983); Lucas and Kates, Cell 7:397-405 (1976)). Using fractions of the cytoplasm or nucleus as cytoplasts and karyoplasts, respectively, avoids the mixed genotype problems described above. Unfortunately, these methods have not resulted in the generation of cells that can proliferate for long periods of time in culture.

Finally, a comprehensive reprogramming has been achieved through the technique of somatic cell nuclear transfer leading to the generation of adult animals from an adult cell nucleus transferred into an enucleated oocyte. This technique has been demonstrated in sheep, goats, cows, pigs and mice (Wilmut et al., Nature 385:810-813 (1997); Kato et al., Science 282:2095-2098 (1998); Wells et al., Biol. Reprod. 60:996-1005 (1999); Kubota, et al., Proc. Natl. Acad. Sci. USA 97:990-995 (2000); Wakayama et al., Nature 394:369-374 (1998); Wakayama and Yanagimachi, Nature Genetics 22:127-128 (1999)). This technology is the subject of many issued patents and patent submissions. The transfer of a nucleus to an enucleated oocyte of the same species generates a “reconstructed embryo” which can be implanted into a foster mother and taken to term. This process is called “reproductive cloning,” because it results in a completely reproduced organism. A variation, “therapeutic cloning,” has been put forth as a way to provide specific cell types customized to individual human patients for uses such as replacement or supplementation of diseased cells, tissue or organs.

Therapeutic cloning has been proposed (reviewed by Colman and Kind, Trends in Biotechnology, 18, 192-196, 2000) to produce cloned embryos from which human embryonic stem (ES) cells can be made. The specific human ES cells could then be cultured in vitro and induced to differentiate, instead of implanted into a foster mother as in reproductive cloning. Although this technology is currently being perfected, a major hurdle is the provision of sufficient human oocytes as nuclear transfer recipients. Nuclear transfer is still a very inefficient procedure. An estimated 200 oocytes are needed to produce one human ES cell line. Therefore huge logistical and ethical problems are present.

It would be advantageous if non-human recipient cells were available, instead. Recently, using nuclear donors from a variety of species in combination with enucleated bovine oocytes (Dominko et al., Biol. Reprod 6:1496-1502 (1999); also see WO 98/07841 (1998)), the inventors have shown that reconstructed embryos can develop at least to the blastocyst stage. However, it is not clear whether these embryos or cells derived from them retain any further proliferative potential. A potential barrier to further proliferation might be that mitochondria of the recipient oocyte are found in the animal resulting from nuclear transfer. Mitochondria from one genome appear to be incompatible with a nuclear genome from even closely related species, thus resulting in the non-viability of the “cybrids” (hybrid cells containing the nucleus from one species and the cytoplasm from another; reviewed in Colman and Kind supra). The relative ratios of oocyte cytoplasm to nuclear donor cell cytoplasm may effect this problem.

The patent application WO 99/45100, entitled “Embryonic or Stem Cell Lines Produced by Cross Species Nuclear Transplantation” attempted to address these problems by producing an embryo from cross species nuclear transplantation, from which pluripotent cells are then produced. This procedure allows a much higher relative contribution of donor cytoplasm to the hybrid, thus, greatly enhancing the long term proliferative potential of the hybrids formed. To date, though, there have been no reports of survival of cross species, nuclear transfer (NT) embryos beyond the blastocyst stages (100-200 cells). This short survival time could be because most if not all the mitochondria are maternally derived in NT embryos (Sheils et al., Nature 399:316-317 (1999)). Long term survival of most hybrid cells made from combinations of the cytoplasts of one species and the nucleus from a different species cannot usually survive in the absence of mitochondria from the cytoplast donor (Kenyon and Morales, Proc. Natl. Acad. Sci. USA 94:9131-9135 (1997)).

The present invention solves this problem and provides the means for making “personalized” tissue and organs for patients in need thereof. The invention, hence, solves the problems of heteroplasmic incompatibility as well as the risk of cross-species contamination that is posed to the society at large by xeno-transplantation.

### SUMMARY OF THE INVENTION

The invention is of the production of pluripotent cells using cytoplast fragments obtained from either whole enucleated oocytes or whole, enucleated fertilized zygotes. These cytoplasts may be obtained from species which do not present insurmountable financial or ethical hurdles to their collection. The cytoplast fragments are fused with nuclear donors of either the same species or another species. These nuclear donors are either whole cells or karyoplasts. Once the cytoplast and nuclear donor are fused, the hybrid is maintained in an undifferentiated state, so that the genetic information of the nuclear donor is reprogrammed into that of an undifferentiated cell. When this is achieved, the hybrid can then be induced to differentiate into the desired cell type. Ultimately, this will allow the production of differentiated cells of any cell type, in any species, which can be used for transplants.

The invention allows for minimization of heteroplasmic incompatibility in tissue and organ transplantation by using only a fragment of the oocyte cytoplasm to induce dedifferentiation of a nuclear donor, instead of the entire enucleated oocyte. Additional steps, such as inactivation of the oocyte mitochondrial replication and supplementation with mitochondria of the nuclear donor species, are also used to avoid the problems of cross-species nuclear transfer, without the ethical problems of producing an embryo. Unexpectedly, this method also addresses the difficulties encountered in deriving embryonic stem cells from mammalian species apart from mice and possibly some primates, see U.S. Pat. No. 5,843,780 (1998), since the cells generated by this new method may have properties similar to animal ES cells, in that they will be pluripotent.

In a first aspect, the invention provides for the production of a pluripotent cell which is the result of the fusion of a mammalian cytoplast fragment derived from an oocyte or fertilized zygote with a cell or a karyoplast (the “nuclear donor”) taken from any mammalian species. The cytoplast donor can be from any mammalian species, but preferably from one of mouse, rat, rabbit, sheep, goat, pig, or most preferably, cow. It is an object of the invention to provide an economical and ethical means of pluripotent cell production from humans where human oocytes or embryos are not needed to derive such cells. However, a preferred method, in the absence of these concerns, is where the donor karyoplast and cytoplast fragments are obtained from the same species.

In a second aspect of the invention, the viability of mitochondria in the cytoplast are compromised by the use of inhibitors of mitochondrial function or replication. Alternatively, cytoplasts which contain congenital mitochondrial lesions are chosen.

In a third aspect of the invention, the mitochondrial content of the cells produced in the first and second aspect of the inveniton is supplemented by the introduction of mitochondria, preferably from the same source as the donor. Most preferrably, the introduced mitochondria are introduced by fusion of the cells with platelets or enucleated lymphocytes from the same source as the donor. However, mitochondria prepared according to standard procedures from any of a variety of cell types may be used. Optionally, such mitochondrial populations would be microinjected into cells of the invention.

In a fourth aspect of the invention, donor cells produced according to the first, second or third aspects of the invention are transfected with genes encoding proteinaceous factors whose normal roles is to modulate transcription and/or replication of mitochondrial DNA. The added genes are preferrably obtained from the same species providing the cytoplast.

In a fifth aspect of the invention, reprogramming of the nucleus in cells prepared according to the first, second, third, or fourth aspects of the invention is facilitated by the transfection of the cells with genes whose products can enhance chromatin remodelling. The genes can be stably integrated into the cells or preferably, transiently transfected.

In a sixth aspect of the invention, reprogramming in cells made according to aspects one, two, three or four of the invention, is facilitated by the use of chemical or biologically derived agents known to cause gene reactivation. Examples of such reagents are trichostatin A, or other histone deacetylation inhibitors. Furthermore, compounds which catalyze histone deacetylation such as butyrate, are also used to promote gene activation by loosening nucleosome-nucleosome interactions which allow access of transcription factors.

In a seventh aspect of the invention, cells made according to any one of the above aspects of the invention are propagated in culture under conditions designed to discourage differentiation. Such conditions may include the addition of various media supplements (e.g., LIF, steel factor) as well as growth (e.g., bFGF, GCT44).

An eight aspect of the invention provides for the differentiation of cells cultured according to the seventh aspect of the invention, into desired cell types. Preferably, such differentiation is achieved by the addition of cocktails of growth factors and other components formulated to ensure the differentiation into specific cell types.

In a ninth aspect of the invention, differentiation of cells made according to aspects one to six of the invention, is assisted by the transfection of genes encoding transcription factors or other specific gene activators.

A tenth aspect of the invention provides for transfection of genes either before (in primary cell cultures), after (in pluripotent cell cultures) hybrid-derived cells are produced, or after hybrid-derived cells are induced to differentiate. Preferred genes are those designed to correct genetic defects or supply cells with the capacity to produce a desired protein, enzyme, enzyme product, cellular component, etc., that may be activated constitutively, upon induction by a trans-activator, or upon transplant into the appropriate milieu. It is the object of the invention for such genetic modifications to be either targeted or heterologous.

In an eleventh aspect of the invention, a method is provided for selecting fusion products on a background of potentially unfused cells. Similarly, a method is provided for selecting hybrid cells with a normal karyotype against a background of aneuploid, hybrid cells. The method utilizes cell tracker probes and nucleic acids encoding fluorescent proteins in order to mark and identify the cytoplasts fragments, nuclear donors cells, and nucleic acids of the invention by color and/or fluorescence. Selection of fused products and cells having normal karyotypes is accomplished using a cell sorter device. This method allows for enrichment of fused cells and fused cells having normal karyotypes.

### BRIEF DESCRIPTION OF THE DRAWINGS/FIGURES

FIG. 1. Fragmented bovine cytoplasts produced by vortexing in cytochalasin B. Cytoplasts containing the endogenous chromosomes have been removed using micromanipulation with Hoechst 33342 fluorescent dye.

FIG. 2. DIC (A) and epifluorescent (B) micrographs of fragmented bovine cytoplasts following fusion with porcine fetal fibroblasts. Reconstructed hybrid cells were cultured for 18 hours after fusion without receiving an activation stimulus. Total magnification 200×.

FIG. 3. Micrographs of fixed reconstructed cell hybrids after 48 hours of culture with BrdU. (A) Hybrids were fixed using 50 mM glycine in 70% ethanol, pH 2.0 and labeled with FITC-conjugated anti-BrdU monoclonal antibody. (B) The same hybrids in (A) were counterstained with DAPI to visualize the nuclei. Total magnification 200×.

FIG. 4. DIC (A) and epifluorescent (B) micrographs of fragmented bovine cytoplasts following fusion with porcine fetal fibroblasts. Reconstructed hybrid cells were cultured for 7 days after fusion and received an activation stimulus after fusion. The hybrids were cultured in SOF in 30 μl drops under mineral oil without fibroblast feeder cells. Total magnification 200×.

FIG. 5. Phase contrast (A), DIC (B) and epifluorescent (C) micrographs of fragmented bovine cytoplasts following fusion with porcine fetal fibroblasts. Reconstructed hybrid cells were cultured for 7 days after fusion and received an activation stimulus after fusion. The hybrids were cultured in SOF in 30 μl drops under mineral oil with fibroblast feeder cells. Total magnification 400× in (A) and 200× in (B) and (C).

FIG. 6. Characterization of cytoplasts from bovine MII arrested oocytes. (A) Cytoplasts fractionated from 3 bovine oocytes. (B) Arrow indicates a lysed cytoplast which occurs in 1-3 of cytoplasts. (C) Size comparison between an intact zona free oocyte and fractionated cytoplasts. (D) Distribution of mitochondria labeled with MitoTracker before fractionation. (E) Distribution of mitochondria after fractionation. (F) Same cytoplasts in (E) labeled with non-specific DNA dye showing distribution of RNA.

FIG. 7. FACS analysis of hybrid cell sort with selection for Hoechst 3342. Two peaks are shown. Peak D corresponds to monulcleate hybrid cells. Peak B corresponds to multinucleate hybrid cells.

### DETAILED DESCRIPTION OF THE INVENTION

The invention relates to the preparation of pluripotent cells which can be directed to differentiate into different cell lineages. These pluripotent cells are produced by a method in which an oocyte or fertilized zygote is fragmented to produce cytoplasts which are then fused with somatic or stem cells (or their karyoplasts) from a different species.

The present invention is directed to the production of pluripotent cells using cytoplast fragments of enucleated oocytes. One embodiment of the invention is the cytoplast fragments of enucleated oocytes which can participate in reprogramming of a differentiated cell. Another embodiment of the invention is a method of making these cytoplast fragments of enucleated oocytes. A third embodiment is a pluripotent cell obtained using oocyte cytoplast fragments. A fourth embodiment of the invention is a method to make pluripotent cells using oocyte cytoplast fragments. A detailed description enabling each of these embodiments follows.

In the description that follows, a number of terms conventionally used in the field are utilized extensively. In order to provide a clear and consistent understanding of the specification and the claims, and the scope to be given such terms, the following definitions are provided.

“Autologous” implies identical nuclear genetic identity between donor cells or tissue and those of the recipient.

“Cytoplast fragment,” with regard to the invention, is a fragment of an oocyte or fertilized zygote which is less than the entire cytoplasm of the oocyte and lacks a nucleus or nuclear DNA material. A cytoplast fragment is also enclosed by a membrane, either the plasma membrane or an artificial membrane. Cytoplasts can also be made of other non-oocyte or zygote cells.

“Embryonic stem (ES)” cells are rapidly dividing cultured cells isolated from cultured embryos which retain in culture the ability to give rise in vivo to all the cell types which comprise the adult animal including the germ cells.

“Embryonic germ (EG)” cells are generated by the culture of primordial germ cells taken from later stage embryos which retain in culture the ability to give rise in vivo to all the cell types which comprise the adult animal including the germ cells.

“Hybrid cell” refers to the cell immediately formed by the fusion of a unit of cytoplasm formed from the fragmentation of an oocyte or zygote with an intact somatic or stem cell or alternatively a derivative portion of said somatic or stem cell, containing the nucleus.

“Karyoplast” refers to a fragment of a cell containing the chromosomes and nuclear DNA. A karyoplast is surrounded by a membrane, either the nuclear membrane or other natural or artificial membrane.

“Multipotent” implies that a cell is capable, through its progeny, of giving rise to several different cell types found in the adult animal.

“Nuclear transfer” refers to the technique whereby the nucleus/genome of an oocyte, egg, or zygote is substituted by a nucleus taken from (usually) a somatic or stem cell.

“Oocyte” refers to the female germ cell during its progression through meiosis. An MII oocyte refers to an oocyte at the second meiotic metaphase stage of meiosis; an activated oocyte is an MII oocyte which has been activated either by sperm or any of a variety of artificial stimuli, to complete meiosis.

“Pluripotent” implies that a cell is capable, through its progeny, of giving rise to all the cell types which comprise the adult animal including the germ cells. Embryonic stem and embryonic germ cells are pluripotent cells under this definition.

A “reconstructed embryo” is an embryo made by the fusion of an enucleated oocyte with a somatic or ES or EG cell; alternatively, the somatic cell nucleus can be injected into the oocyte.

“Stem cell” describes cells which are able to regenerate themselves and also to give rise to progenitor cells which ultimately will generate cells developmentally restricted to specific lineages.

“Somatic cells” describes all types of cell apart from germ cells, embryonic stem and germ cells (see definitions below), which are present in, or derived from, the embryonic, fetal and adult stages of development.

“Totipotent” implies that a cell is capable, through its progeny, of giving rise to all the cell types which comprise the adult animal including the germ line, as well as any cell types required to nurture the growing embryo and fetus (e.g., trophoblast tissue). In mammals, only the zygote and (in some species) early blastomeres qualify as totipotent. When the term is used to describe a nucleus, it implies that the nucleus is capable, given the appropriate cytoplasmic environment, of supporting the developmental program described above.

“Transgenic” animal or cell refers to animals or cells whose genome has been subject to technical intervention including the addition, removal, or modification of genetic information.

“Zygote” refers to a fertilized one-cell embryo.

Production of Cytoplast Fragments

The species used for cytoplast donors will vary depending on the reprogramming potential of cytoplasts from a particular species and on the cost of obtaining oocytes from it. Successful production of live offspring using somatic cell nuclear transfer demonstrates that the cytoplasm of oocytes from cows, mice, sheep, goats, and pigs are capable of conferring totipotency onto a single nucleus from a somatic donor cell of the same species. In vivo matured oocytes from these species may be recovered from the oviducts of either naturally or artificially synchronized female donor animals (techniques are widely known to those skilled in the art; instructions for many procedures are documented in Transgenic Animal Technology: A Laboratory Handbook, Pinkert, C. A., ed., Academic Press, San Diego, Calif. (1994)).

Alternatively, oocytes obtained from human and livestock species may be matured in vitro. In the case of livestock, antral follicles present in ovaries obtained at slaughter are aspirated and immature oocytes are induced to undergo in vitro maturation by incubation with an appropriate mixture of culture medium, nutrients, and hormones (reviewed by Trounsen et al., Theriogenology 41:57-66 (1994)). Transvaginal oocyte recovery (TVOR (Pieterse et al., Theriogenology 30:751-762 (1988) and in vitro maturation (Susko-Parrish, et al.) have been demonstrated for bovine oocytes from live cows or heifers, though in vivo matured bovine oocytes are expensive and difficult to collect from the animal. Because it is known that there are differences in the viability of embryos depending on whether the oocytes are matured in vivo or in vitro, if in vivoderived oocytes are required, they can readily be obtained from murine, rabbit, sheep, goat, pig, and primate species. Preferably, oocytes are obtained from a source in which the starting material can be screened and controlled for the absence of specific known pathogens. Examples of such animals include various types of cattle, ungulates (ie. sheep, goats, pigs, horses) lagomorphs (ie. rabbits), and rodents.

Three major populations of oocytes can be used for cytoplast preparation: 1) unactivated, high MPF, MII arrested oocyte cytoplasts, 2) activated, low MPF (interphase) oocytes, and 3) aged, unactivated, low MPF oocyte cytoplasts. Each of these cell cycle stages has been shown to have certain advantages for reprogramming (Barnes et al., Mol. Reprod. Dev. 36:3341 (1993); Campbell et al., Biol Reprod 49:933-942 (1993)). A preferred embodiment involves the use of high MPF oocytes for cytoplasts. Activation of oocytes prior to cytoplast preparation can be accomplished using a variety of established protocols, including inter alia, electrical pulse, ionomycin/DMAP, disintegrin, calcium ionophore, cycloheximide, strontium, sperm factor, and sperm (reviewed by Campbell, Cloning 1:3-15 (1999)).

The endogenous maternal genomic DNA in the nucleus is removed either before or after cytoplast production. The nucleus is removed using either micromanipulation or bulk removal procedures (i.e. centrifugation in an appropriate gradient such as Percholl® in the presence of a microfilament inhibitor such as cytochalasin B).

In a preferred method for cytoplast production, the zona pellucida is removed by incubation with an effective concentration of an appropriate enzyme (e.g., pronase) or acidified Tyrodes solution. Alternatively, the zona pellucida may be removed by mechanical means using micromanipulation, followed by incubation in an appropriate concentration of a microfilament inhibitor such as cytochalasin B and vortexing. The speed and duration of the vortexing are determined such that optimal cytoplast size is achieved. For example, speed 10 on a Vortex Genie 2 for 7 seconds for porcine or rabbit oocytes (see Ex. 1 and 2), or max speed on a Vortex Genie 2 for 3 minutes for bovine oocytes (see Ex. 3) can be utilized. Alternative methods of cytoplast production include micropipetting in the presence of an effective concentration of microfilament inhibitor as well as repeated mechanical aspiration of specific amounts of cytoplasm (e.g. enough to yield 10-50 cytoplast fragments per oocyte), using micromanipulation procedures or slicing the cytoplast in the presence of a microfilament inhibitor using a suitable tool.

Enucleated oocyte cytoplast fragments are fractions of an oocyte constituting less than the entire cytoplasm. Mammalian oocytes are fractionated into enucleated cytoplasts of an appropriate size such that enough intracellular material is maintained in each fragment to induce nuclear envelope breakdown and chromosome condensation of the nuclear donor in the production of pluripotent hybrid cells. On the other hand, the amount of intracellular material in each cytoplast should not be so high that the number of cytoplasts available from one oocyte is limited to one. For the most efficient use of donor oocytes, it is preferred that more than 10 and up to 50 cytoplast fragments are obtained from the factionation of each oocyte. It is anticipated that smaller volume cytoplasts will present fewer problems of mitochondrial incompatibility. While cytoplast fragments of various sizes can be used, a general size range is about 120 μm to about 5 μm in diameter. In a preferred embodiment, the size of the cytoplast fragments can range from about 100 μm to about 10 μm in diameter. In another preferred embodiment the size of the cytoplast fragments can range from about 90 μm to about 20 μm in diameter. In another preferred embodiment, the size of the cytoplast fragments can range from about 80 μm to about 30 μm in diameter. In another preferred embodiment, the size of the cytoplast fragments can range from about 70 μm to about 30 μm in diameter. In another embodiment, the size of the cytoplast fragments can range from about 60 μm to about 30 μm in diameter. In another preferred embodiment, the size of the cytoplast fragments can range from about 60 μm to about 20 μm in diameter. In more preferred embodiment, the size of the cytoplast fragments can range from about 50 μm to about 10 μm in diameter. In a more preferred embodiment, the size of the cytoplast fragments can range from about 20 μm to about 30 μm in diameter. In the most preferred embodiment, the size of the cytoplast fragments is about 25 μm in diameter. Cytoplasts can be separated according to their size by size fractionation in an appropriate gradient or by using a cell sorter, and cytoplasts of different diameters examined as recipients in hybrid construction. On average, between 15 and 50 cytoplasts are produced by a single oocyte.

The viability of cytoplasts is expected to be 24-48 hours based on enucleated cytoplast produced from cultured somatic cells (Goldman et al., Proc Natl Acad Sci USA 70:750-754 (1973)). Some loss of cytoplasts is expected due to small size or lysis. Distribution of cellular organelles and other cell components is expected to be uniform among cytoplasts (Cohen et al., Theriogenology 43:129-140 (1995)).

Production of Nuclear Donor

In principal, any differentiated somatic or stem cell can be used for hybrid production. Preferred cell types include those that have been shown to be reprogrammable in nuclear transfer. Somatic cells that have been used for nuclear transfer (NT) to produce live offspring include skin fibroblasts (goats: Baguisi et al., Nature Biotech 17:456-461 (1999)), leukocytes (cattle: Galli et al., Cloning 1:161-170 (1999)), granulosa and cumulus cells (mice: Wakayama et al., Nature 394:369-374 (1998); cattle: Wells et al., Biol Reprod 60:996-1005 (1999)), oviductal epithelium (cattle: Kato et al., Science 282:2095-2098 (1998)), mammary gland cells (sheep: Wilmut et al., Nature 385, 810-813 (1997)), and fetal fibroblasts (Schneike et al., Science 278:2130-2133 (1997)). Other non-limiting examples of cells suitable for NT include keratinocytes, hepatocytes, respiratory epithelial cells, neuronal cells, C34+ stem cells, and granulocytes. Further preferred cell types are those easily obtained by non-invasive biopsy procedures. Examples include skin fibroblasts and mononuclear peripheral blood cells. It would be within the skill of the ordinary practitioner to determine other cells suitable as nuclear donors within the scope of the invention.

Cells are harvested from the donor organism using routine biopsy and cell isolation procedures. Cells are either used fresh or cultured and allowed to proliferate in vitro to amplify them for use and cryopreservation. In a preferred embodiment the cell cycle stage of the nuclear donor is matched to the cell cycle stage of the recipient cytoplast For metaphase I cytoplasts (high MPF) it is preferred that the cells are synchronized in G0/G1 so that upon activation the appropriate nuclear ploidy is maintained due to appropriate DNA replication. The cells may be synchronized in G0/G1 by various methods available in the art, such as culture in low serum concentration, culture in the presence of trichostatin A (a histone deacetylase inhibitor), or by contact inhibition.

If karyoplasts are used as nuclear donors, they may be prepared by various methods from interphase cells. For example, karyoplasts can be prepared by centrifuging a cell suspension through a 12.5-25% non-linear Ficoll density gradient (Ohara et al., J. Immunol. Meth 45:239-248 (1981)) in the presence of 101 g/ml cytochalasin B. The fraction corresponding to 17.5-25% Ficoll is collected and the karyoplasts are purified further using a continuous BSA (bovine serum albumin) sedimentation gradient.

If the donor nucleus is derived from a cytoplasm-deficient karyoplast, then mitochondrial supplementation is preferred. Methods for introducing mitochondria into mitochondrial deficient cells are available in the art (King and Attardi, Meth. Enzymol. 264:304-334 (1996)). For example, enucleated cells may be fused to reconstructed hybrids. Preferred enucleated cells are those naturally occurring such as blood platelets. In a more preferred embodiment, the enucleated mitochondrial donor cells are blood platelets from the same individual as the karyoplast nuclear donor.

Production of Hybrid Cells (HDCs)

There are many techniques available in the art that can be used to induce fusion of one cell type to another, even across species (for example, rat-mouse: Krondahl et al., Proc. Natl. Acad Sci. USA 74:606-609 (1977); human-mouse: Hightower et al., Proc. Natl. Acad Sci. USA 80:5310-5314 (1983); chicken-human: Rao, Exp. Cell Res. 102:25-30 (1976); chicken-hamster: Dubbs and Kit, Som. Cell Gen. 2:11-19 (1976); chicken-rat: Scheer et al., J. Cell. Biol. 97:1641-1643 (1983)). Examples of these cell fusion methods include, inter alia, the use of inactivated Sendai virus, electrical stimulation, polyethylene glycol (PEG), high pH-low osmolarity medium, hemagglutinin (HA), and liposomes. A preferred method is one that maximizes the efficiency of hybrid production without adversely affecting the viability of the hybrids formed. For example, exposure to an optimal concentration of PEG in culture medium or exposure to electrical stimulation using optimal parameters in an optimal medium. The parameters for the preferred embodiment for PEG mediated fusion is accomplished by exposing the cells/karyoplasts and cytoplasts to about 40-50% PEG for about one minute. Using these conditions, virtually all cytoplasts that are in contact with the donor cell or karyoplast will fuse.

The most preferred embodiment involves electrical fusion. Electrical fusion is performed by placing the cytoplasts and cells/karyoplasts in a appropriate fusion medium in a chamber between 2 electrodes attached to a high voltage DC pulse generator. Fusion is induced by applying one or multiple high voltage/short duration DC pulses. A preferred method is where the fusion medium consists of 0.3 M manitol, 0.05 mM MgCl₂, 0.1 mg/ml polyvinyl alcohol, the DC voltage is 1.25 kV/cm, and the couplets are allowed to equilibrate in the fusion medium for 10 minutes prior to fusion.

The most preferred embodiment for electrical fusion, the fusion medium includes 0.28 M mannitol, 0.05 mM MgCl2, 0.1 mg/ml polyvinyl alcohol, and a DC voltage of 2.0-2.5 Kv/cm. The ratio of the number of cytoplasts to cells/karyoplasts is optimized to reduce the number of multiploid fusions while maximizing the number of diploid fusions. A preferred range of ratios of cytoplasts to karyoplasts or cells is about 0.01:1 to about 0.1:1, with the most preferred ratio being 0.1:1. More important is the concentration of cells (karyoplasts) per volume of fusion medium. A range from 20,000-100,000 cells per 20 ul volume is preferred, with the most preferred cell concentration being 80,000 cells per 20 ul of fusion medium in a 2 mm fusion chamber.

In order for subsequent development and proliferation to occur post-fusion, the newly fused hybrids must be activated to simulate cell cycle progression similar to that induced by sperm at fertilization. There are many artificial activation methods available in the art which have been shown to induce both development of parthenotes and nuclear transfer couplets. For example activation may be achieved by electrical pulse (Kono et al., Theriogenology 33:569-576 (1989)); Prochazka et al., J. Reprod Fert. 96:725-734 (1992)), ionomycin/DMAP (Susko-Parrish et al., Dev Biol 166:729-739 (1994)), ethanol (Nagai, Gamete Res 16:243-249 (1987)), cytochalasin/cychloheximide (Presicce and Yang, Mol. Reprod. Dev. 37:61-68 (1994)), strontium (Oneil et al., Mol. Reprod. Dev. 30:214-219 (1991)), adenophostin (Sato et al., Biol. Reprod 58:867-873 (1998)), disintegrin RGD peptide (Campbell et al., Proc Park City Utah Conference Abst #7 (1998)), DDT/thimerosal (Machaty et al., Biol. Reprod 56:921-930 (1997)), and sperm factor (Swann, Development 110:1295-1302 (1990); Stice and Robl, Mol. Reprod Dev. 25:272-280 (1990)). All of these methods have been shown to induce at least some specific biochemical effect that is similar to that observed during natural fertilization, followed by parthenogenic development to at least the blastocyst stage in vitro. In a preferred embodiment, the activation stimulus is customized for the cytoplast species being used by experimental optimization.

The activation stimulus can be delivered to the cytoplasts before, during or after fusion is induced. In a preferred embodiment, the activation stimulus is delivered such that the cell stage of the cytoplast is matched with that of the donor cell/kayoplast. By matching the cell cycles, anuploidy in the resultant hybrid cells is minimized or eliminated. In a more preferred embodiment, the cell/karyoplast is synchronized in G1/G0 and the cytoplast is maintained at metaphase arrest (the natural arrest point for mature ovulated oocytes awaiting fertilization). In this instance enhanced nuclear remodeling occurs because the diploid donor chromosomes are induced to undergo nuclear envelope breakdown (NEVBD) and to condense in the recipient cytoplasm NEVBD and chromatin condensation allow molecules from the oocyte cytoplast fragment to remodel the nuclear donor chromatin, thus “erasing” the chromatin structure native to the differentiated cell (i.e. the “memory” of the differentiated state is erased). Upon subsequent activation the donor DNA is induced to enter S-phase and replicate the “erased” genome in accordance with the timing of the first cell cycle of a newly fertilized oocyte.

In addition, pluripotent cells are produced by fusion of nuclear donor cells or karyoplasts with cytoplast fragments derived from same species as the nuclear donor. Preferred embodiments include the use of bovine, porcine, ovine, caprine, rabbit, and primate differentiated cells as nuclear donors and oocyte cytoplasts from the same species, respectively.

Inhibition of Replication of Cytoplast Donor Mitochondria and Supplementation of Mitochondria from Nuclear Donor Species

A preferred embodiment involves making the mitochondria of the oocyte cytoplast fragment replication incompetent by incubation with an appropriate inhibitor of mitochondrial DNA replication. This will ensure homoplasmy, that is, a homogenous source of mitochondria in the cytoplasm, in the hybrids for the mitochondria from the donor cell. An inhibitor of mitochondrial DNA replication is the DNA intercalating dye ethidium bromide (King and Attardi, Meth Enzymol 264:304-334 (1996)). Cellular respiration is maintained when mitochondrial replication is inhibited by supplementation with glucose, pyruvate, and uridine (King and Attardi, Meth Enzymol 264:304-334 (1996)).

To minimize detrimental effects that mitochondrial heteroplasmy, that is, mitochondria from divergent sources or species, may have on the proliferating hybrids, mitochondria derived from the nuclear donor species are used to supplement those in the hybrid cell population. Cells derived from the nuclear donor animal are enucleated and the remaining enucleated cytoplasts are fused with the hybrid cells. To remove nuclei from adherent cells, the cells are centrifuged in the presence of a suitable microfilament inhibitor such as cytochalasin B. The nuclei pinch off and migrate to the bottom of the tube, leaving the cytoplasm plus mitochondria The enucleated cytoplasts are then fused with hybrid cells using common fusion protocols such as electrical fusion or polyethylene glycol. In a preferred embodiment, this is accomplished by fusion of hybrids with already enucleated, mitochondria-rich cells from the nuclear donor, such as blood-derived platelets (King and Attardi, Meth. Enzymol. 264:304-334 (1996)).

Kenyon and Morales, Proc. Natl. Acad. Sci. USA 94:9131-9135 (1997)) showed that in trans-species hybrid cells, there seemed to be an incompatibility between the nucleus of one species and the mitochondria of another. If it is difficult to proliferate hybrid cells made from certain species combinations, donor cells might be transfected with genes encoding important mitochondrial maintenance factors like the transcription factor, mtTFA (Larsson et al., Nat. Genet. 18:231-236 (1998)). Reference to other factors can be found in Shade and Clayton, Ann. Rev. Biochem. 66:409435 (1997)). Methods of cell transfection are well known in the art.

Enhancement of Reprogramming Hybrid Cells

Hyperacetylation of lysine residues located on the N-terminal tail of histone core proteins is associated with gene activation and transcription (Almouzni et al., Dev. Biol. 165:654-669 (1994)). Histone acetylation is also associated with the heritability of chromatin structure through mitosis. To facilitate a chromatin structure in the hybrid that is more easily remodeled by the cytoplast fragment, genes expressed in the nuclear donor can be down-regulated and switched off. To achieve this, nuclear donor cells can be transiently transfected with DNA constructs encoding appropriate modulators of gene expression and chromatin structure. In a preferred embodiment, the gene encoding historic deacetylase is transfected into the donor cells at a time prior to hybrid production such that the chromatin of the nuclear donor cell becomes transcriptionally compromised. This effect causes the nuclear donor cell to lose its memory of being a differentiated cell, suitably priming its chromatin structure to take on the structure that is dictated by the oocyte derived cytoplast fragment. Other such genes include, inter alia, Xenopus nucleoplasmin and its mammalian equivalent (Chen et al., 28:1033-1089 (1989)).

Activation of Gene Transcription in Hybrid Cells

Once reconstructed populations of hybrid cells (HDCs) are established, it may be desirable to assist the genome in activation of DNA transcription. This is accomplished by culturing the hybrid cells (HDCs) in medium supplemented with compounds known to induce DNA transcription, such as histone deacetylases inhibitors. In a preferred embodiment, hybrid cells are cultured in the presence of histone deacetylase inhibitors. Examples of histone deacetlyase inhibitors are butyrate and trichostatin A. More preferred is a compound that does not have inhibitory effects on cellular function other than reversible inhibition of histone deacetylase, for example trichostatin A (Almouzni et al., supra (1994)).

Inhibition of Differentiation of Hybrid Cells

The activated hybrids are placed in a culture medium that is appropriate to support development and proliferation while maintaining the de-differentiated state. It is known in the art that when maintained on embryonic fibroblasts in culture, embryonic stem cells retain their totipotential capacity in generating cells of all lineages. Mouse, monkey, and human stem cells can be grown in culture for extended period of time (reviewed by Thomson and Marshall, Curr. Top. Dev. Biol. 38:133-165 (1998)) and remain undifferentiated under specific culture conditions. It is preferred that the culture medium is supplemented with growth factors and cytokines that will maintain the undifferentiated state. Examples of such de-differentiating factors include LIF, Steel factor, and conditioned medium from embryonic fibroblast cultures. Ultimately, the culture medium used depends on the species of cell/karyoplast donor since the growth and maintenance signals provided by the medium components provide gene transcription and regulation signals to that genome. For example, culture conditions known in the art to permit proliferation, while preventing differentiation, for human and monkey embryonic stem cells (Thompson et al., Science 282:1145-1147 (1998); Thompson et al., Proc. Natl. Acad. Sci. USA 92:7844-7848 (1998)) are used when human cells/karyoplasts are used as donors.

In a preferred embodiment, the hybrids are cultured using mitotically inactivated fibroblasts as feeder cells. The feeder layer is made by culturing primary embryonic fibroblasts to about 80% confluence then arresting further growth potential using a mitogenic inactivating agent such as mitomycin-C. The hybrids are then seeded onto this feeder layer in DMEM supplemented with an appropriate serum concentration and other growth factors that support maintenance of an undifferentiated state. The undifferentiated state of embryonic stem cells can be monitored using a variety of cell surface markers. For example, cell surface expression of alkaline phosphatase is common to pluripotent ES cells from mice, non-human primates, and humans. In a preferred embodiment, the hybrids are cultured on a monkey fibroblast feeder layer in DMEM with 20% heat inactivated fetal bovine serum, 0.1 mMβ-mercaptoethanol, 1.0 mM glutamine, 1% non-essential amino acids, and 1000 units/ml recombinant human LIF.

In another embodiment, the hybrid cells (HDCs) are maintained in the undifferentiated state by prior transfection of donor cells with a selectable marker, such as toxin resistance gene, under control of a promoter with expression restricted to undifferentiated cells. Therefore, when the toxin is added to the medium, only the undifferentiated cells will survive. A preferred scheme for such selection is to transfect an oct4-neo DNA construct (McWhir et al., Nature Genet 14:223-226 (1996)), and to grow the hybrids in the presence of geneticin (G418), preventing survival of any differentiated cells. Furthermore, the transgene may be flanked with lox-p sites to permit removal of the expression cassette in cells before transplantation.

As an additional means to maintain an undifferentiated state, the hybrid cells (HDCs) growing on fibroblast feeder layers, are supplemented with GCT44 factor (human yolk sac teratoma cell factor), a factor shown to be beneficial in the maintenance of human EC cell lines (Roach et al., Eur. Urol. 23:82-88 (1993)).

Differentiation of Hybrid Cells to Specific Lineages

Differentiation of hybrid cells (HDCs) to a specific lineage is accomplished by removing the hybrid cells from culture conditions intended to prevent differentiation and supplementing the culture conditions with agents known to induce differentiation of embryonic stem cells into that lineage (reviewed in Fuchs and Segre, Cell 100:143-155 (2000)). Such differentiated cell types include neural cell (oligodendrocytes, astrocytes, dopaminergic neurons), hematopoietic cells (macrophages, erythrocytes), and muscle cells (skeletal, heart vascular smooth muscle).

Hybrids are induced to form neural-like cells by plating them in a defined medium containing a neural pathway differentiation signal such as retinoic acid. Glial cell precursors are induced to differentiate into two distinct populations, oligodendrocytes or astrocytes, by sequential culture in fibroblast growth factor 2 (FGF2), followed by a mixture FGF2 plus epidermal growth factor (EGF), and finally a mixture of FGF2 and platelet-derived growth factor (PDGF). Development of erythrocytes from dedifferentiated hybrid cells is accomplished using c-kit plus erythropoietin. Macrophages are made using a cocktail of macrophage colony stimulating factor (M-CSF), interleukin 1, and interleukin. Differentiation of cultured hybrids to obtain adipocytes is done by culturing in appropriate levels of retinoic acid, insulin, and tri-iodothyronine. Heart vascular smooth muscle cells are produced by culturing hybrids in retinoic acid plus dibutyryl cyclic AMP. Endodermal cells obtained by differentiating hybrids are induced to become pancreatic cell precursors by exposing them to medium conditioned with cells obtained from the pancreatic bud. Differentiation into neural precursors or skeletal myoblasts can be induced by culturing hybrid cells in medium supplemented with retinoic acid or dimethyl sulfoxide, respectively (Dinsmore et al., Cell Transplantation 2:131-143 (1996)).

Differentiation of cells into specific cell types made according to previous aspects of the invention may also be assisted by the transfection of genes encoding transcription factors or other specific gene activators. Such cloned factors have been effective in converting fibroblasts into myoblasts (Myo D: Davis et al., Cell 51:987-1000 (1987)) or in converting fibroblasts (PPAR gamma: Tontonoz et al, Cell 79:147-1156 (1994)) and myoblasts [PPAR gamma and C/EBP alpha: Hu et al., Proc. Natl. Acad. Sci. USA 92:9856-9860 (1995)) into adipocytes.

Production of Genetically Modified Pluripotent Cells

This aspect of the invention relates primarily, but is not limited to human medicinal applications. In order to achieve a modified biological effect of the hybrid cells (HDC's), genes are transfected either before (in primary cell cultures), after (in pluripotent cell cultures) hybrid cells are produced, or after hybrid cells are induced to differentiate. Preferred genes are those designed to correct genetic defects or supply cells with the capacity to produce a desired protein, enzyme, enzyme product, cellular component, or deliver a therapeutic benefit to a specific tissue niche, etc. The gene expression is activated constitutively, upon induction by a trans-activator, or upon transplant into the appropriate milieu. The genetic modifications are either site-specific (targeted) or not (heterologous). In a preferred embodiment, the primary cells are transfected. Thus, the desired genetic modifications are made in early passage cells which reduces the amount in vitro culture for the hybrid cells (HDC's). It is preferred that the desired structural gene be placed under operative control of a promoter suitable for the ultimate cell type desired. There are many tissue-specific and constitutive promoters and methods of transfecting cells in targeted and non-targeted loci available in the art. Corrections of genetic mutations such as those found in sickle cell anemia (β-globin) and diabetes (insulin) are examples of targeted strategies to fix defects in the donor cells. Furthermore, genes may be added to cells (which are subsequently made pluripotent) to compensate for a deficiency of a certain cell type that leads to disease.

A further embodiment involves the use of cells genetically modified to inhibit tissue rejection associated with xenotransplantation of cells. For example, one strategy to combat Parkinson's disease and diabetes is to transplant pig neural and pancreatic islet cells, respectively, from pigs into humans. These approaches are fraught with rejection of the pig tissue by the host immune system. In a preferred embodiment, primary pig cells (such as fibroblasts) are modified to remove the primary xenoantigen (α-1-3 galactosyltransferase. These cells are then made pluripotent, induced to differentiate into neural or pancreatic cells by the methods described in the invention, and used in cell therapy applications for Parkinson's Disease or diabetes, respectively.

In a preferred embodiment the cells from the same species into which the transplant will occur, are genetically modified, fused with cytoplast fragments and subsequently differentiated into the desired cell type, as described by the current invention. For example, somatic cells from a patient with sickle cell anemia are transfected with a DNA construct designed to correct the mutation in the β-globin gene. The corrected cells are made pluripotent, and then differentiated into hematopoietic stem cells by methods of the current invention. Finally the hematopoietic stem cells are transplanted back into the patient to repopulate the bone marrow with corrected cells of autologous origin.

### EXAMPLES

### Example 1

### Production of Porcine-porcine Hybrids with Porcine Cytoplasts

Porcine oocytes arrested at metaphase II of the meiotic cycle were aspirated from pre-ovulatory antral follicles obtained from the ovaries of donor gilts superovulated using standard procedures. The zona pellucidae were removed by incubating oocytes in 0.5 mg/ml Pronase (Sigma Chemical Co., St. Louis, Mo.) in Phosphate Buffered Saline (Gibco BRL, Gaithersburg, Md.) for 10 minutes. Oocytes (200 count) were then incubated in 7.5 μg/ml cytochalasin B (Sigma) in NCSU23 medium (Petters and Wells, J. Rreprod Fert. Suppl. 48:61-73 (1993)) modified to be used as a benchtop holding medium for 5 minutes. The NCSU23 medium was modified by deleting all NaHCO₃, adjusting KH₂PO₄ to 0.44 MM, adding 1.34 mM Na₂HPO₄, and compensating for the changes in K and Na by adjusting the NaCl and KCl concentrations accordingly. Optimal cytoplast size (30-40 μm) was obtained by vortexing (Vortex Genie 2, Scientific Industries, Bohemia, N.Y.) the oocytes in a 0.5 ml Eppendorf microcentrifuge tube for 7 seconds using a speed setting of 10. Contact inhibited (100% confluent) porcine fetal fibroblasts were harvested using 0.25% trypsin-EDTA (Gibco BRL), washed in NCSU23-phosphate, then resuspended in fusion medium (0.3 M mannitol, 0.1 mM CaCl₂, 0.05 mM MgCl₂, 0.1 mg/ml PVA) at a concentration of 1.0×10⁶ cells per ml. Roughly 2.0×10⁴ cells, in 20 ul of fusion medium, were placed into a 3.2 mm fusion chamber (Model BT 453, BTX Gentronics, San Diego, Calif.), and the cytoplasts (approx. 6000) were pipetted into the fusion chamber with the cells. Contact between the cells and oocytes was made by sequentially pipetting the cells and cytoplasts while visualizing them using stereomicroscope. Fusion was induced using two 1.25 KVolts/cm DC pulses for 60 μsec each. The couplets were induced to activate 20 minutes later using another two DC pulses at 1.0 kilovolt/cm for 60 μsec each in activation medium SOR2 (0.3 M sorbitol, 0.1 mM Ca-acetate, 0.05 mM Mg-sulfate). The hybrids were washed free from surrounding cells and placed in NCSU23 culture medium at 38.5 C in a humidified atmosphere of 5% CO₂ in air and examined 18 hours later. Hybrids were placed into NCSU23-phosphate medium containing 7.5 μg/ml Hoechst 33342 (Sigma Chemical Co) and evaluated for fusion efficiency and gross chromatin structure using an Olympus IX70 inverted microscope equipped with narrow band UV epifluorescence. A total of 32 cytoplasts were subjected to fusion, 8 of which fused. In 7 of the fused cytoplasts, there was a single intact set of condensed chromosomes suggesting that the cytoplasts had induced nuclear envelope breakdown and chromatin condensation from fusion of a single cell. The other cytoplast contained DNA from multiple cells.

### Example 2

### Production of Porcine-Rabbit Hybrids with Rabbit Cytoplasts

Rabbit oocytes arrested at metaphase II of the meiotic cycle were flushed from the oviducts of superovulated 6 month old New Zealand White rabbits, using standard protocols. Both pronase and acid Tyrode's solution failed to remove the zona pellucida. Therefore, the cytoplasts were made manually by micromanipulation using 7.5 μg/ml cytochalasin B (Sigma Chemical Co.) in NCSU23-phosphate medium. The hybrids were produced as described above for the porcine-porcine hybrids. A total of 46 cytoplasts were prepared from 3 oocytes and 21 of them fused with a single fetal fibroblast. In 20 of the fused cytoplasts, there was a single swollen nuclear structure suggesting that the cytoplasts had activated. Proliferative potential could not be measured in these initial trials since the culture medium did not contain any growth factors or serum. The purpose of this experiment was only to evaluate cytoplast preparation and fusion.

### Example 3

### Production of Hybrids from Porcine Fetal Fibroblasts with Bovine Cytoplasts: Formation of Stem Cell-like Colonies

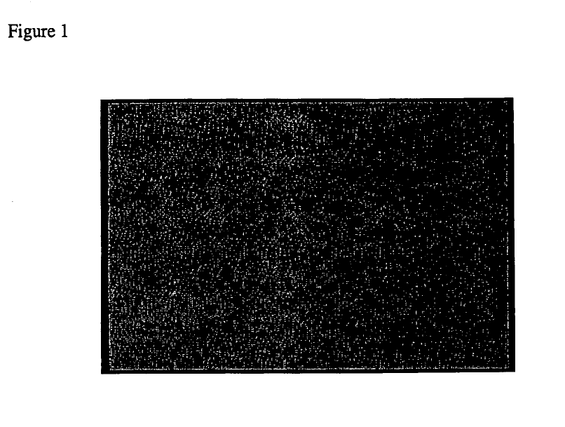

Culture tubes containing bovine cumulus oocyte complexes (COCs) in 5% CO₂-equilibrated maturation medium were shipped overnight in a portable isothermal incubator at 39° C. from the oocyte production laboratory (Genetic Technologies International, Brian, Tex.) to our laboratory. At 18 hours of in vitro maturation, COCs were removed from maturation medium and incubated for 10 minutes in modified phosphate buffered synthetic oviductal fluid (SOF-P) supplemented with 0.3 mg/ml hyaluronidase (Sigma). SOF-P medium was formulated as a benchtop medium for bovine oocytes and embryos to be used outside the incubator. The formulation for SOF (Tervit et al., J. Reprod. Fert. 30:493-497 (1972)) was modified by deleting all sodium bicarbonate, changing the BSA concentration to 1 mg/ml, adjusting KH₂PO₄ to 0.44 mM, adding 1.34 mM Na₂HPO₄, and compensating for the changes in K and Na by adjusting the NaCl and KCl concentrations accordingly. COCs were stripped of the cumulus cells by vortexing (Vortex Genie 2, Scientific Industries, Bohemia, N.Y.) at maximum speed for 3 minutes in 1 ml of medium in a 15 ml conical centrifuge tube (Falcon, Cat # 05-52790). Oocytes were scored for successful completion of nuclear maturation by the presence of a single polar body (PB+) in the perivitellin space using a standard stereornicroscope (Olympus SXH). PB+ oocytes were allowed to recover from hyaluronidase treatment for 1 hour in bicarbonate buffered synthetic oviductal fluid (SOF) supplemented with ½× non-essential and essential amino acids (Gibco BRL, Gaitherburg, Md.) at 38.5° C. and 5% CO₂ in air. The zona pellucidae were removed by incubation in SOF-P with 2 mg/ml pronase (Calbiochem, La Jolla, Calif.) with continuous pipetting. Zona free oocytes were placed in SOF-P microdrops to recover for 20 minutes, then incubated in SOF-P with 5.0 μg/ml cytochalasin B (CB, Calbiochem) and 7.5 μg/ml) Hoechst 33342 (Calbiochem) for 10 minutes. Groups of 2040 oocytes were fragmented by vortexing in 200 μL of SOF-P with CB in a 1.5 ml microcentrifuge tube for 5 -10 seconds. Cytoplast fragments (FIG. 1) were collected and placed in a microdrop of SOF-P and cytoplasts containing the endogenous chromosomes were identified using UV illumination (Olympus IX 70 inverted microscope) and removed using a 22 μm micropipette (Humagen, Charlottesville, Va.) connected to a syringe microinjector (Cell Tram-oil, Eppendorf Scientific, Westbury, N.Y.), and controlled using a micromanipulator (Model # MMN-202D, Narishige, East Meadow, N.Y.). Enucleated cytoplasts were transferred to fresh medium and held until fusion with nucleated cells.

Cells used as nuclear donors were porcine fetal fibroblasts grown to confluence in a 35 mm culture dish containing 2 ml of culture medium. Culture medium consisted of DMEM (Gibco BRL), 10% FCS (Hyclone, Loagn, Utah), and 2.0 mg/ml bFGF (Collaborative Biomedical Products, Bedford, Mass.). Cells were harvested using trypsin/EGTA and suspended in SOF-P. Cells were pelleted by centrifugation at 1600 RPM for 4 minutes, all medium removed and the pellet resuspended in 1.0 ml of fusion medium (0.3 M manitol, 0.05 mM MgCl₂, and 0.1 mg/ml polyvinyl alcohol). The cell concentration was approximately 1.0×10⁶ per ml. Cytoplasts were added to the fusion medium containing the cells and allowed to settle to the bottom of the tube. A mixture of cells and cytoplasts were aspirated from the bottom of the tube and placed into a 3.2 cm fusion chamber (Model BT 453, BTX, Gentronics, San Diego, Calif.) filled with fusion medium. Fusion was induced using a single 1.25 KVolts/cm DC pulse for 60 μsec (Model ECM 2001, BTX). The couplets were washed out of fusion medium into SOF-P with 20% heat inactivated FCS. Couplets were induced to activate by incubation in SOF-P containing 5.9 μM ionomycin (Sigma) for 4 minutes, followed by incubation in SOF containing 2.0 mM dimethylanimopurine (DMAP, Sigma) for 4 hours. Couplets were then cultured in 30 μl drops of SOF under oil at 38.5° C. in humidified atmosphere of 5% CO₂ in air. Unfused porcine fetal fibroblasts were added to the culture to be used as feeder cells.

A group of non-activated hybrid cells was cultured overnight and stained using Hoechst 3342 to evaluate chromatin structure. In addition, a group of activated hybrid cells were cultured in a separate drop without feeder cells in SOF containing 10× the manufacturer's recommendation of bromodeoxy uridine (]3rdU) to assess proliferative potential by assaying them for DNA replication. After 1 week of culture the remaining activated hybrid cells were assessed using DIC, phase contrast, and epi-fluorescence (after staining with 1 μg/ml Hoechst 33342) microscopy.

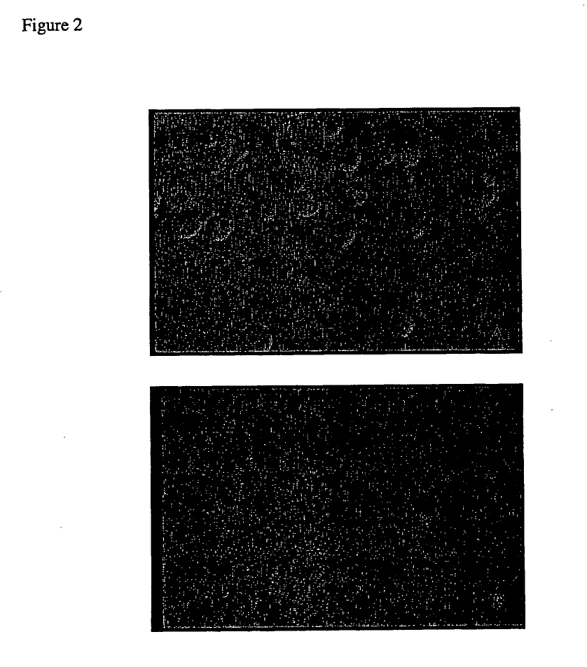

Of the cytoplasts that survived fusion and were not activated with ionomycin and DMAP, all of them contained condensed DNA and an intact nuclear membrane was absent (FIG. 2). This observation suggested that the oocyte fragments were indeed capable of inducing nuclear envelope breakdown and premature chromatin condensation (PCC) on the nuclear donor cell. This is a process known to occur when an intact metaphase 11 stage oocyte is fused S-phase blastomeres (Campbell et al., Biol. Reprod 50:1385-1393, (1994)) and quiescent cells (Wakayama et al., Nature 394:369-374 (1998)).

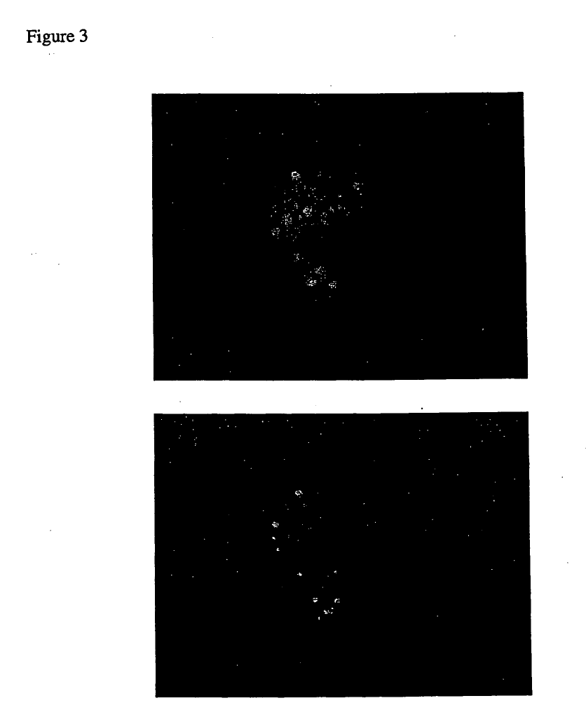

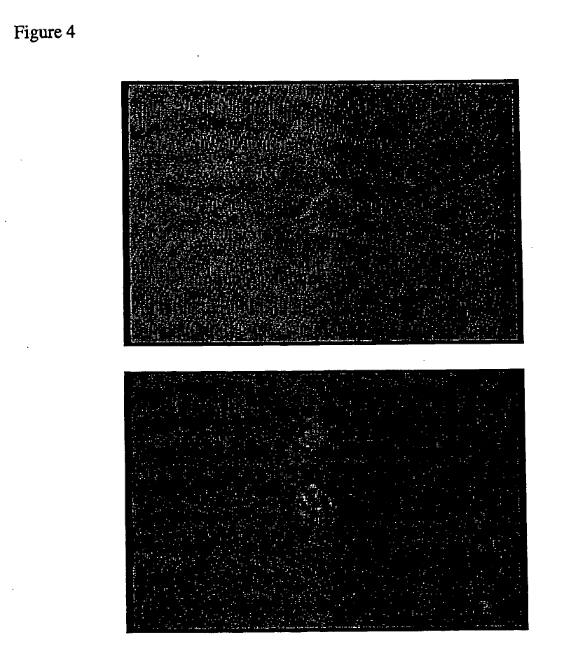

The group of cytoplasts that were cultured for 20 hours after activation in the presence of BrdU with activation were analyzed for evidence of DNA synthesis using a BrdU incorporation immunofluorescent assay (Roche Molecular Biochemicals, Indianapolis, Ind.). An aggregate of hybrid cells of unknown cell number was fixed using 50 mM glycine (Sigma) in 70% ethanol (Sigma), pH 2.0 at −20 C. BrdU incorporation assay was performed using a FITC-labeled anti-BrdU monoclonal antibody according to the manufacturer's instructions. Samples were counterstained for bulk DNA content with DAPI (Sigma) and the presence of both FICT. and DAPI was assessed by epifluorescence using an Olympus AX-70 research microscope equipped with appropriate excitation filters (Chroma Technology Corp., Brattleboro, Vt.). Digital grayscale images were recorded for each fluorochrome using an LAR Astrocam (Model TE3/A/S) cooled CCD camera. A composite image was constructed by psuedocoloring the grayscale image from both fluorochromes (blue for DAPI and green for FICT.) using LAR Ultra view Spatial Imaging Module (v 2.2.1) image analysis software. Most of the cells in the aggregate stained green indicating that DNA synthesis had occurred in the activated reconstructed hybrid cells (FIG. 3). After culture for 7 days without feeder cells, the hybrids aggregated with one another and appeared to proliferate as an embroid body or mass, possibly indicating the ability to differentiate (FIG. 4).

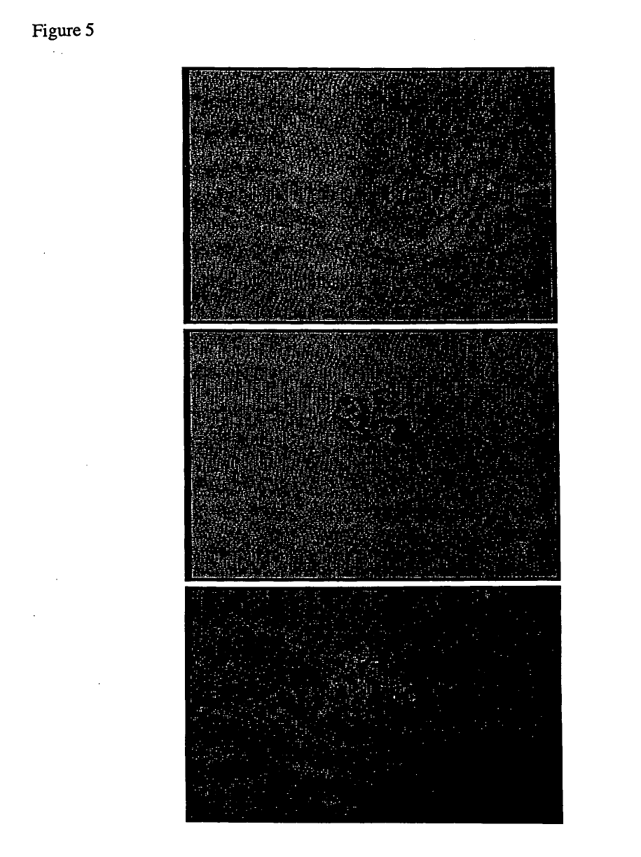

The unfused fibroblasts attached to the bottom of the culture dish in small aggregates of cells. Associated with these small fibroblast colonies, cell colonies of entirely different morphology were also present at much higher cell number. These colonies were characterized by a large nucleus to cytoplasm ratio, formation of tight aggregates of cells rising above the culture dish surface, and small round nuclear morphology. The morphological characteristics of these cells resembled that of embryonic stem cells (FIG. 5). Since all of the bovine cytoplasts containing any bovine DNA were removed prior to fusion, the cells of ES-like morphology had to have arisen from reconstructed hybrid cells between the porcine fetal fibroblasts and enucleated bovine cytoplasts.

### Example 4

### Preparation and Characterization of Bovine Oocyte Cytoplasts

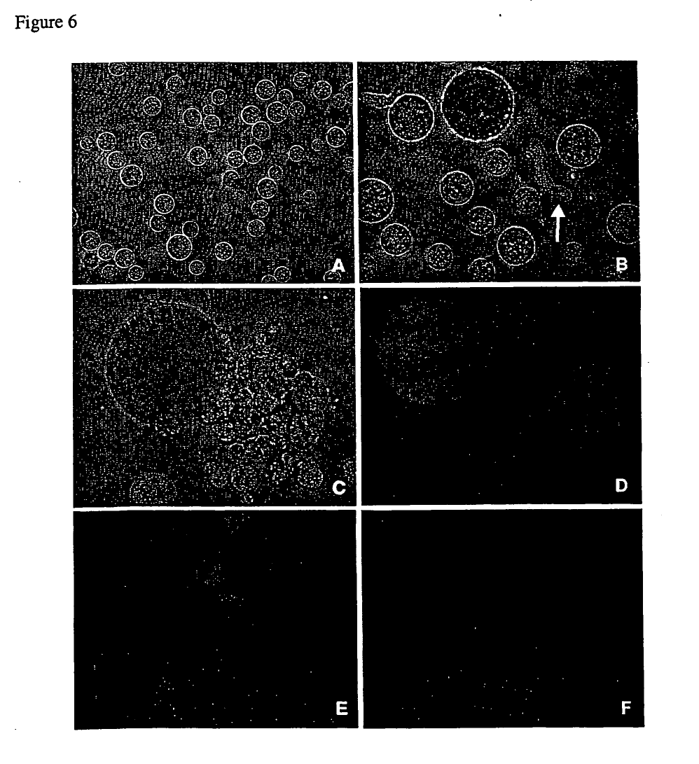

All culture media and cytoplast preparation methods were the same as those described in Example 3. Cytoplast fragments were prepared from bovine oocytes (FIG. 6A). The results indicated that incubation of zona-free oocytes in cytochalasin B allowed for their fractionation by vortexing without significant lysis (2-4%), and enabled the correlation of vortexing time with the desired cytoplast fragment size. Visualization of fractionated oocytes pre-labeled with a vital mitochondrial dye (MitoTracker, Molecular Probes, Eugene, Oreg.) and DNA stain (Syto 16, Molecular Probes) indicated that the cytoplasmic fragments retained comparable amounts of live mitochondria and RNAs (FIG. 6, E and F). This result confirmed the assumption that oocyte cellular components were evenly distributed among cytoplasts after fractionation, which suggested that a high proportion of the cytoplasts were of similar quality with respect to these markers. In addition, the cytoplast fragments retained their size, shape, and membrane integrity for at least 48 hours (data not shown), and survived cryopreservation after thawing.

### Example 5

### Generation of Cardiomycytes (beating muscle cells) from Hybrid-Derived Cell Preparations Demonstrates Reprogramming

Bovine cytoplasts were prepared using the same method as in Example 4 above. Cytoplast fragments obtained from 500 oocytes (approx. 15,000 cytoplasts) were added to 1 ml fusion medium (0.28 M mannitol in water) containing 1×10⁶ cells and allowed to settle to the bottom of the tube. A mixture of cells and cytoplasts were aspirated from the bottom of the tube and placed into a 3.2 cm fusion chamber (Model BT 453, BTX, Gentronics, San Diego, Calif.) filled with fusion medium (50 ul). Fusion was induced using a single 1.25 KVolts/cm DC pulse for 60 μsec (Model ECM 2001, BTX). The couplets were washed out of fusion medium into SOF-P with 20% heat inactivated FCS. Couplets were induced to activate by incubation in SOF-P containing 5.9 μM ionomycin (Sigma) for 4 minutes, followed by incubation in SOF containing 2.0 mM dimethylanimopurine (DMAP, Sigma) for 4 hours. The couplets were subsequently washed out of DMAP and plated onto feeder layers of y-irradiated primary mouse embryonic fibroblasts in stem cell medium (high glucose DMEM w/o pyruvate, 20% FBS, 2 nM glutamine, 1% non-essential amino acids, 0.1 mM beta-mercaptoethanol, and 1000 units/ml recombinant human LIF).

The cells used as nuclear donors (BSFF-GFP cells) were bovine fetal fibroblasts from the Brown Swiss breed and were transgenic for Green Florescent Protein (GFP), where the GFP gene was under control of the constitutive elongation factor promoter (EF-1α), to allow the visualization of HDC-derived colonies in the presence of the feeder layer. After 7 days culture of the HDCs on feeder layers, numerous GFP (+) colonies were observed with stem cell-like morphology. One GFP (+) colony had a large lobe of cells (roughly 30% of the colony) that were beating rhythmically. This colony continued to beat for 2 weeks. These myocardial-like cells are similar to morphologies obtained from spontaneous differentiation of mouse ES cells, and are an early indication that these bovine hybrid-derived cell colonies are pluripotent. This example demonstrates the usefulness of the methods of the invention in reprogramming cells.

### Example 6

### Use of Florescent Activated Cell Sorting (FACS) to Enrich for Fusion Products (Hybrid Derived Cells)

Two separate experiments were performed which utilize cell sorting and enrichment methods for the generation and selection of hybrid-derived cells, and are outlined below. In both experiments, florescent Cell-Tracker dyes (Molecular Probes) were used to stain cytoplasts, in order to follow the cytoplasm during the generation of cytoplast/cell fusion products. Experiment I used bovine fetal fibroblasts as the nuclear donor and a vital DNA-staining dye (Hoechst 33342), while Experiment 2 utilized cells transgenic for Green Florescent Protein (GFP) as a means of marking the nucleus of the donor cell.

Steps 1, 3, 5, and 6 are common to both Exps. 1 & 2.

Step 1: Oocyte/Cytoplast Preparation

Bovine oocytes, aspirated from slaughterhouse ovaries, were received by overnight shipment from a commercial oocyte provider (Ovagenix; Genetic Technologies International, San Angelo, Tex.). They were shipped in maturation medium and were expected to be at meiotic metaphase II after 24 hours in maturation medium. Oocytes were removed from the maturation medium (M199 with Earles Salts with L-glutamine and sodium bicarbonate (Life Technologies); with 10% FBS (Hyclone), 2 u/ml bFSH (Sioux Biochemical), 1.5 u/ml bLH(Sioux Biochemical)), washed in FHM with phenol red (Specialty Media) and incubated for 5 minutes in hylauronidase (0.3 mg/ml). Cumulus cells were removed by rapid vortexing for 3 minutes. The oocytes were washed and rested for 5 minutes in FHM.

Oocytes were incubated for 30 mins in 2.5 uM Cell Tracker Dye (Molecular Probes C-2925 (green) for Experiment 1, below or orange for experiment 2, below) to stain the cytoplasm. They were washed and allowed to recover for 15-30 minutes in FHM. Zonae pellucidae were removed/dissolved by a brief (2-6 minute) incubation in pronase (5 mg/ml Sigma) and polyvinyl pyrrolidone (0.5 mg/ml; Sigma) in PBS. Zona-free oocytes were washed and allowed to recover for 30-60 minutes in FHM.

To fragment the oocytes into cytoplasts (roughly 40 per oocyte), zona-free oocytes were incubated in 20 ul fusion medium (0.3 M mannitol in water) with cytochalasin B (7.5 ug/ml; Sigma) for 10 minutes and vortexed for 3-30 seconds. The cytoplasts were ready for fusion to nuclear donor cells.

Step 2: Preparation of Nuclear Donor Cells

Experiment 1:

Bovine Brown Swiss Fetal Fibroblast (BSFF) cells were seeded at least 3 days prior to their use for this experiment. They were cultured in DMEM with non-essential amino acids (0.1 mM; Specialty Media) and 20% FBS (Specialty Media) until 24-36 hours before use and then cultured in DMEM with non-essential amino acids without serum (serum starved).

BSFF cells were removed from the culture dish by removing media, washing with PBS, then incubating in trypsin-EDTA (Life Technologies) for 2-5 minutes. Cells were centrifuged and the pellet resuspended in fusion medium containing Hoechst 33342 (7.5 ug/ml; Sigma) for 5 minutes. BSFF cells were counted and aliquots of 40,000-100,000 cells were placed in 0.5 ml microfuge tubes, centrifuged and resuspended in 20 ul of fusion medium.

Experiment 2:

BSFF cells were transfected by a standard lipofection method (Lipofectamine, Gibco) with a Green Fluorescent Protein (GFP) gene, under control of the constitutive elongation factor (EF)-α promoter. The resulting transgenic cells, called BSFF-GFP cells, were seeded at least 3 days prior to their use for this experiment. They were cultured in DMEM (Specialty Media) with non-essential amino acids (0.1 mM; Specialty Media) and 20% FBS (Specialty Media) until 24-36 hours before use and then cultured in DMEM with non-essential amino acids (0.1 mM) without serum (serum starved).

BSFF-GFP cells were removed from the culture dish by removing media, washing with PBS, then incubating in trypsin-EDTA (Life Technologies) for 2-5 minutes. Cells were centrifuged and the pellet resuspended in fusion medium (0.3 M mannitol in water). BSFF-GFP cells were counted and aliquots of 40,000-100,000 cells were placed in 0.5 ml microfuge tubes, centrifuged and resuspended in 20 ul of fusion medium.

Step 3: Fusion

Cytoplasts and cells were mixed in a 0.5 ml microfuge tube and aliquots of 20 ul were placed in the fusion chamber (BTX P/N 450; 2 mm electrode gap; electrodes on glass slide OR 2 mm gap cuvette electrode). Two pulses were applied, pulse length was 40-80 us, and pulse strength was 40-100 V (1-2.5 kV/cm). The contents of the fusion chamber were removed immediately after fusion and placed in FHM. Optimum parameters were: 80,000 BSFF cells and up to 10,000 cytoplasts/20 ul fusion volume, 60 us pulse length, 2 pulses, 2.4 kV/cm pulse strength. Fusion rates of up to 97% were achieved using these optimal parameters.

Step 4: Fluorescent-Activated Cell Sorting (FACS)

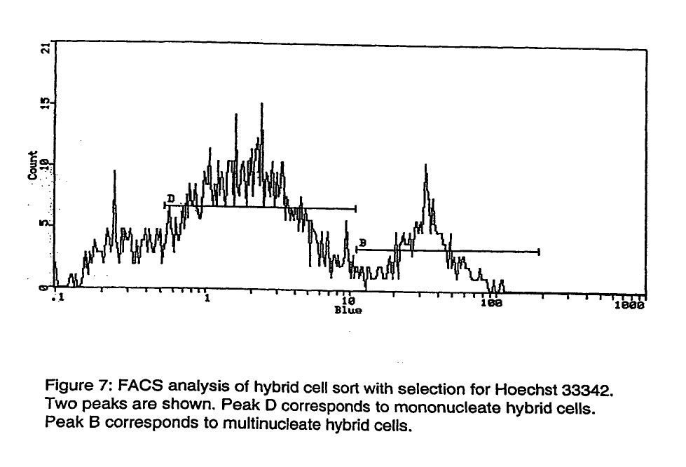

Fusion products were sorted on a Becton Dickenson cell sorter.
a. Using BSFF cells from Experiment 1 above: Fusion products (hybrid cells) were sorted from unfused BSFF cells by selecting firstly for green Cell-Tracker dye. All cytoplasts (fused and unfused) were selected. The products of this sort were sorted a second time, with selection for Hoechst 33342 blue-stained DNA. Thus the population of cells was enriched for cytoplasts (green) fused with BSFF cells (blue). Results from the second sort show that there were two fluorescent peaks visible (FIG. 7). The first peak (D) corresponds to mononucleate hybrid cells and the second peak (B) corresponds to multinucleate hybrid cells. Therefore, in addition to enriching for fusion products, the FACS sort provides a method for sorting aneuploid hybrid cells away from those with a normal karyotype.
b. Using BSFF-GFP cells from Experiment 2 above: Fusion products (hybrid cells) were sorted firstly from unfused BSFF cells by selecting for orange Cell-Tracker dye. All cytoplasts (fused and unfused) were selected. The FACS was also able to sort green GFP positive BSFF-GFP cells from the population, providing a method for enriching for BSFF-GFP cells. Using this double-dye sorting method, it was possible to significantly enrich for fusion products (orange cytoplasm with a green nucleus), without having to do a secondary stain with a DNA-specific dye such as Hoechst.
   5. Activation

Hybrid cells were activated 30-60 minutes after the FACS sort using either: 4 minutes in Ionomycin (25 uM in FHM; Sigma) then 4 hours in DMAP (2 mM in culture medium; Calbiochem); OR: 6 minutes in 7% ethanol in FHM, then 1 hour in cycloheximide (7.5 ug/ml; Calbiochem) and cytochalasin D (10 ug/ml; Sigma) in culture medium, then 3 hours in cycloheximide (7.5 ug/ml) in culture medium.

6. Culture

Hybrid cells were cultured either on mitomycin C inactivated mouse embryonic fibroblast feeder layers (Specialty Media) in stem cell medium (DMEM with 20% FBS, 1 mM non-essential amino acids, 1 mM L-glutamine (Sigma), 0.1 mM beta-mercaptoethanol (Sigma); or on untreated tissue culture plates in G1/G2 (IVF Scientific) sequential medium.

1-47. (canceled)

48. A method of generating a hybrid mammalian cell comprising:
(a) preparing more than one cytoplast fragment from a mammalian metaphase 11 oocyte or fertilized zygote wherein the amount of cytoplasm in the cytoplast fragment is less than the amount of cytoplasm in the mammalian oocyte or fertilized zygote;
(b) obtaining a nuclear donor cell or karyoplast taken from a mammal;
(c) combining one cytoplast fragment of step a) with the nuclear donor cell or karyoplast of step b) to produce a hybrid mammalian cell; and
(d) if an oocyte is used in step (a), then activating the oocyte before, during or after step (c).

49. The method of claim 48, wherein the cytoplast fragment is produced by vortexing the mammalian oocyte or fertilized zygote.

50. The method of claim 48, wherein the mammalian oocyte or fertilized zygote is surrounded by a zona pellucida and wherein the zona pellucida is removed prior to step (a).

51. The method of claim 48, wherein the mammalian oocyte-fertilized zygote, or resulting fragment thereof is enucleated.

52. The method of claim 48, wherein the mammalian oocyte is matured in vitro or in vivo.

53. The method of claim 48, wherein the mammalian oocyte is selected from the group consisting of: an activated, low maturation promotion factor oocyte; an aged, unactivated, low maturation promotion factor oocyte; and an unactivated, high maturation promotion factor, metaphase II oocyte.

54. The method of claim 48, wherein the cytoplast fragment is from a different species from that of the nuclear donor.

55. The method of claim 48, wherein the cytoplast fragment is from the same species as that of the nuclear donor.

56. The method of claim 48, wherein the cytoplast fragment is prepared from a mammalian oocyte or fertilized zygote taken from a non-human mammalian species.

57. The method of claim 48, wherein the nuclear donor cell is selected from the group consisting of fibroblasts, skin fibroblasts, leukocytes, granulosa cells, cumulus cells, oviductal epithelium, mammary gland cells, fetal fibroblasts, keratinocytes, hepatocytes, respiratory epithelial cells, neuronal cells, CD34+stem cells, granulocytes, and mononuclear peripheral blood cells.

58. The method of claim 48, further comprising maintaining the pluipotency by placing the cell in a culture media that supports development and proliferation while maintaining the dedifferentiated state.

59. The method of claim 48, wherein the combining of the cytoplast fragment with the nuclear donor is mediated by electrical fusion, chemical fusion, viruses, liposomes or cell surface proteins.

60. The method of claim 48, wherein the nuclear donor is from an embryonic, fetal, or adult cell, or an embryonic, fetal, or adult karyoplast.

61. The method of claim 48, wherein the nuclear donor is a diploid cell or is taken from a diploid cell.

62. The method of claim 48, wherein the nuclear donor is from a stem cell, or differentiated or undifferentiated somatic cell.

63. The method of claim 48, wherein the nuclear donor has been genetically modified.

64. The method of claim 48, further comprising the step of establishing a population of hybrid cells derived from the hybrid cell.

65. The method of claim 48, wherein more than 10 cytoplast fragments are prepared.

66. A method for reprogramming mammalian cells comprising:
(a) preparing more than one cytoplast fragment from a mammalian mammalian metaphase II oocyte or fertilized zygote wherein the amount of cytoplasm in the cytoplast fragment is less than the amount of cytoplasm in the mammalian oocyte or fertilized zygote;
(b) obtaining a nuclear donor cell or karyoplast taken from a mammal; and
(c) combining one cytoplast fragment of step a) with the nuclear donor cell or karyoplast of step b) to produce a reprogrammed mammalian cell; and
(d) if an oocyte is used in step (a), then activating the oocyte before, during or after step (c).

67. The method of claim 66, wherein the reprogrammed mammalian cell is a cardiomycyte.

68. A method of generating a hybrid mammalian cell comprising:
(a) preparing more than one cytoplast fragment from a mammalian metaphase II oocyte or fertilized zygote wherein the amount of cytoplasm in the cytoplast fragment is less than the amount of cytoplasm in the mammalian oocyte or fertilized zygote;
(b) obtaining nuclear donor cell or karyoplast taken from a mammal; and
(c) combining one cytoplast fragment of step a) with the nuclear donor cell or karyoplast of step (b) to produce a hybrid mammalian cell; and
(d) if an oocyte is used in step (a), then activating the oocyte before, during or after step (c).
